(19) United States
(12) Patent Application Publication
(10) Pub. No.: US 2024/0282435 A1
(43) Pub. Date: Aug. 22, 2024

(54) WORK INFORMATION PROCESSING DEVICE, METHOD AND PROGRAM

(71) Applicant: NIPPON TELEGRAPH AND TELEPHONE CORPORATION, Tokyo (JP)

(72) Inventors: Narimune MATSUMURA, Musashino-shi, Tokyo (JP); Jiro NAGAO, Musashino-shi, Tokyo (JP); Daiki SATO, Musashino-shi, Tokyo (JP)

(73) Assignee: NIPPON TELEGRAPH AND TELEPHONE CORPORATION, Tokyo (JP)

(21) Appl. No.: 18/571,257

(22) PCT Filed: Jun. 21, 2021
(86) PCT No.: PCT/JP2021/023461

§ 371 Date: Dec. 18, 2023

(51) Int. Cl. G16H 40/20; G06F 3/01

(52) U.S. Cl. CPC G16H 40/20; G06F 3/011

(57) ABSTRACT

A work information processing device according to an embodiment of the present invention receives an inter-human body communication signal output from an inter-human body communication system in response to a touch between a first person and a second person in work, detects a time-series position of the touch based on the received inter-human body communication signal, and generates touch time-series data indicating a detection result. A work section in which the work is performed is detected from the touch time-series data, the touch time-series data included in the detected work section is extracted, a feature amount of the touch in the work section is detected based on the extracted touch time-series data of the work section, and a type of work is estimated based on the detected feature amount of the touch. For each work, work history information including information indicating the estimated type of work is generated.

### TECHNICAL FIELD

An aspect of the present invention relates to a work information processing device used in a type of industry in which people touch each other to perform work, such as a medical site or a care site, and a work information processing method and a program executed by the device.

### BACKGROUND ART

In recent years, as one of short-range radio communication technologies, an inter-human body communication technology has attracted attention. The inter-human body communication technology is a communication technology of using the individual bodies of people as a transmission path and transmitting information through the bodies between terminals carried by the people when the people touch each other (for example, see PTL 1 and NPL 1 and NPL 2).

According to this technology, for example, when medical personnel such as a doctor or a nurse or a worker such as a caregiver touch the body of a work target such as a patient or a care target to perform various types of work at a medical site or a care site, it is possible to record which worker touches which work target as a history.

### CITATION LIST

### Patent Literature

[PTL 1] Japanese Patent No. 4069061

### Non Patent Literature

[NPL 1] Chizuru Yamao et al. “Business Strategy Utilizing “Human Body Vicinity Electric Field Communication Technology “RedTacton” NTT Technical Review, January 2010
[NPL 2] Mitsuru Shinagawa “Communication Technology Using Human Body as Transmission Line”, Journal of the Institute of Image Information and Television Engineers, vol. 63, No. 11, pp. 1525 to 1530, 2009

### SUMMARY OF INVENTION

### Technical Problem

Incidentally, in the inter-human body communication technology, who touches who can be detected, but a type of performed work may not be determined. Therefore, for example, parties are required to write information such as kinds of work in management files of a work history or the like in person after the work, and thus workloads are large.

The present invention has been devised in view of the foregoing circumstances, and an object of the present invention is to provide a technology capable of recording a type of work in addition to presence or absence of the work and accordingly reducing a workload of an operator.

### Solution to Problem

To solve the foregoing problems, according to an aspect of the present invention, in a work information processing device or method, an inter-human body communication signal output from an inter-human body communication system is received in response to a touch between a first person and a second person in work, a position of the touch in a time series is detected based on the received inter-human body communication signal, and a touch time-series data indicating a result of the detection is generated. Then, a work section in which the work is performed is detected from the touch time-series data, and the touch time-series data included in the detected work section is extracted, a feature amount of the touch in the work section is detected based on the touch time-series data included in the extracted work section, and a type of work is estimated based on the detected feature amount of the touch. Then, for each item of work, work history information including information indicating the estimated type of work is generated and stored.

According to another aspect of the present invention, a feature amount of a touch in work is detected based on an inter-human body communication signal output from an inter-human body communication system in response to a touch between persons in the work, the type of work is estimated based on the feature amount of the touch, and work history information including information indicating the type of work is generated.

### Advantageous Effects of Invention

Accordingly, a type of work can be estimated based on an inter-human body communication signal and can be stored as a work history. Thus, it is not necessary for a worker to record the type of work in the work history by himself or herself, and the work load is considerably reduced.

That is, according to one embodiment of the present invention, it is possible to provide a technology for recording a type of work in addition to presence or absence of work and reducing a workload of the worker.

### BRIEF DESCRIPTION OF DRAWINGS

FIG. 1 is a diagram illustrating an example of a configuration of a work management system including a work information processing device according to an embodiment of the present invention.

FIG. 2 is a block diagram illustrating an example of a hardware configuration of the work information processing device according to the embodiment of the present invention.

FIG. 3 is a block diagram illustrating an example of a software configuration of the work information processing device according to the embodiment of the present invention.

FIG. 4 is a flowchart for illustrating a processing procedure and processing content of work information processing performed by the work information processing device illustrated in FIGS. 2 and 3.

FIG. 5 is a diagram illustrating an example of a work segmentation detection result and an example of a work generation result of a touch time histogram by work information processing shown in FIG. 4.

FIG. 6 is a diagram illustrating an example of work history information obtained through the work information processing illustrated in FIG. 4.

### DESCRIPTION OF EMBODIMENTS

Hereinafter, an embodiment according to the present invention will be described with reference to the drawings.

### Embodiment

### (Configuration Example)

### (1) System

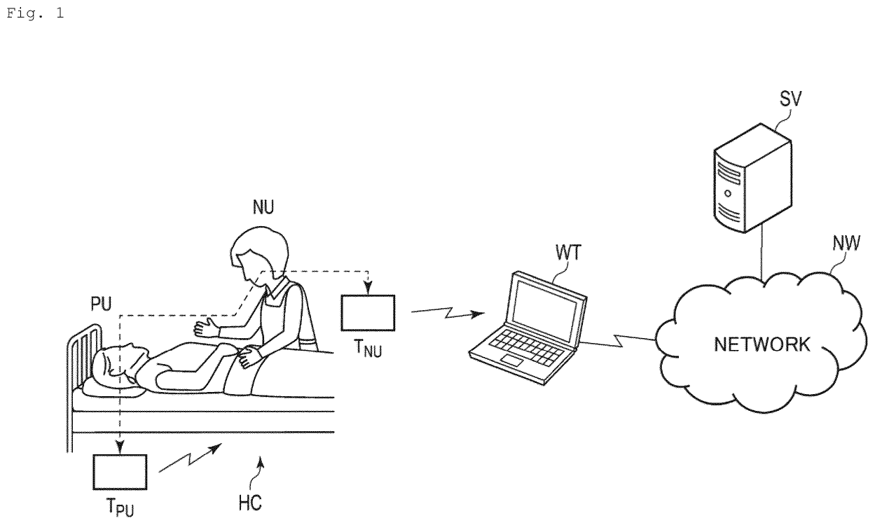

FIG. 1 is a diagram illustrating an example of a configuration of a work management system including a work information processing device according to an embodiment of the present invention.

The work management system according to the embodiment includes an inter-human body communication system HC and a work information processing device WT that receives an inter-human body communication signal transmitted from the inter-human body communication system HC. The work information processing device WT can transmit information to, for example, a server device SV on a cloud via a network NW.

In the inter-human body communication system HC, for example, communication terminals T_{NU} and T_{PU} are disposed in a worker NU such as a nurse or a caregiver and a work target PU such as a patient or a care target, respectively. When the worker NU touches the work target PU, both or either one of the communication terminals T_{NU} and T_{PU} transmits an inter-human body communication signal transmitted by using the body of the worker NU and the body of the work target PU as a transmission line between the communication terminals T_{NU} and T_{PU} to the work information processing device WT. As illustrated In FIG. 1, to facilitate description, the communication terminals T_{NU} and T_{PU} are located away from the worker NU and the work target PU, but the communication terminals T_{NU} and T_{PU} are actually disposed to be in contact with the bodies of the work target NU and the work target PU.

That is, the inter-human body communication system HC transmits a communication signal indicating a time-series change of touch of the worker NU to the work target PU during nursing or care work. In the communication signals, a worker ID and a work target ID for identifying the worker NU and the work target PU are inserted in the communication terminals T_{NU} and T_{PU}.

The server device SV is used, for example, to download a program required by the work information processing device WT, to share some of functions of the work information processing device WT, and to collectively manage work history information generated by the work information processing device WT along with work history information for other work targets PU.

The network NW includes, for example, a wide area network including the Internet and an access network for accessing the wide area network. As the access network, a local area network (LAN) using wired or wireless communication or a public network using wired or wireless communication may be used, but another communication line such as a CATV line may be used.

### (2) Work Information Processing Device WT

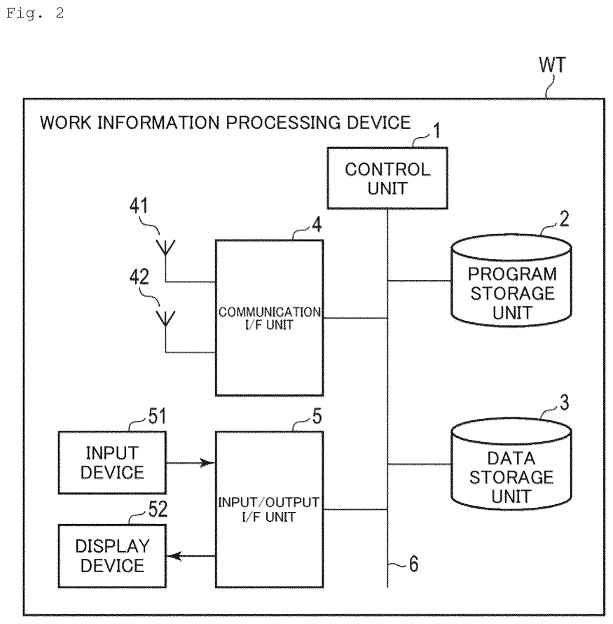

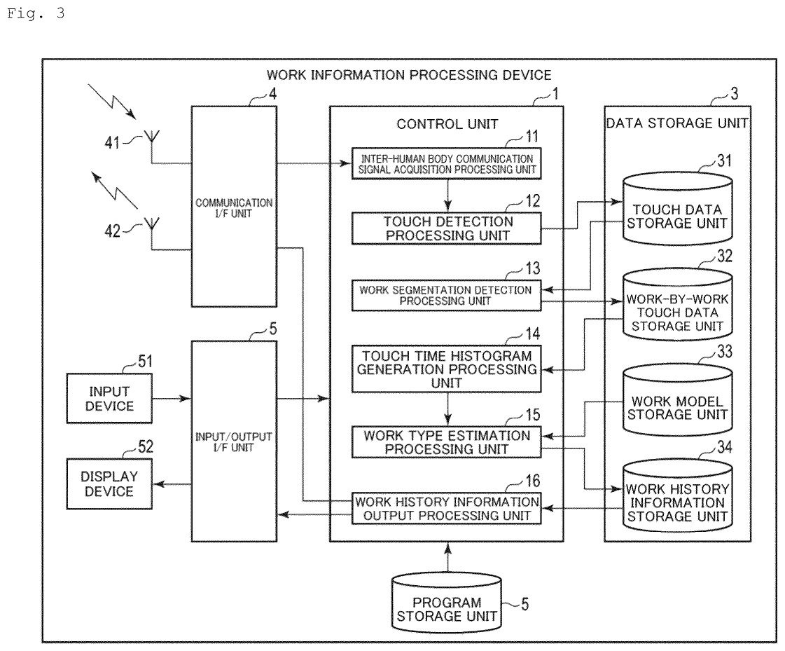

FIGS. 2 and 3 are block diagrams illustrating an example of hardware and software configurations of the work information processing device WT according to the embodiment of the present invention.

The work information processing device WT is configured by, for example, a personal computer and includes a control unit 1 in which a hardware processor such as a central processing unit (CPU) is used. A storage unit that includes a program storage unit 2 and a data storage unit 3, a communication I/F unit 4, and an input/output I/F unit 5 are connected to the control unit 1 through a bus 6.

The communication I/F unit 4 receives the inter-human body communication signal transmitted from the inter-human body communication system HC under the control of the control unit 1, and transmits and receives information data to and from the server device SV by using a communication protocol defined by the network NW. As a radio interface for receiving the inter-human body communication signal transmitted from the inter-human body communication system HC, a radio interface adopting a small power radio data communication standard such as Bluetooth (registered trademark) or Wi-Fi (registered trademark) is used. As a communication interface for transmitting and receiving information data to and from the server device SV, a communication interface used in a local area network (LAN) using a wired or wireless way, or a radio communication interface used in a mobile communication network such as 4G or 5G is used.

An input device 51 and a display device 52 are connected to the input/output I/F unit 5. The input device 51 includes, for example, a keyboard or a mouse, and is used by the worker NU inputting information necessary for work management and inputting an output instruction of work history information. The display device 52 includes, for example, a liquid crystal display or an organic EL display and is used to display work management information such as work history information.

The program storage unit 2 stores application programs necessary to perform various control processes according to the embodiment of the present invention in addition to middleware such as an operating system (OS), for example, by combining a nonvolatile memory such as a hard disk drive (HDD) and a solid state drive (SSD) capable of performing writing and reading frequently and a nonvolatile memory such as a read-only memory (ROM) serving as a storage medium.

The data storage unit 3 includes a touch data storage unit 31, a work-by-work touch data storage unit 32, a work model storage unit 33, and a work history information storage unit 34 as main storage units, for example, by combining a nonvolatile memory such as an HDD and an SSD capable of performing writing and reading frequently and a volatile memory such as a random access memory (RAM) serving as storage media.

The touch data storage unit 31 is used to store time-series data indicating a touch period detected from an inter-human body communication signal transmitted from the inter-human body communication system HC.

The work-by-work touch data storage unit 32 is used to store work-by-work touch time-series data generated by segmenting the time-series data indicating a touch period for each work section.

The work model storage unit 33 is used to store learned data of a work model generated in pre-learning.

The work history information storage unit 34 is used to store work history information including a plurality of information elements necessary for management of work for each work. The information elements of the work history information include, for example, a type of work, a worker ID, a work target ID, and a start time and an end time of a work.

As a processing function according to one embodiment of the present invention, the control unit 1 includes an inter-human body communication signal acquisition processing unit 11, a touch detection processing unit 12, a work segmentation detection processing unit 13, a touch time histogram generation processing unit 14, a work type estimation processing unit 15, and a work history information output processing unit 16. These processing function units 11 to 16 are each implemented by causing the hardware processor of the control unit 1 to execute a program stored in the program storage unit 2.

The inter-human body communication signal acquisition processing unit 11 performs processes of receiving inter-human body communication signals transmitted from communication terminals T_{NU} and T_{PU} of the inter-human body communication system HC via the communication I/F unit 4, converting the received inter-human body communication signals into digital data, and then transferring the digital data to the touch detection processing unit 12.

The touch detection processing unit 12 performs processes of detecting a period of a touch from the inter-human body communication signals, generating time-series data (hereinafter referred to as touch time-series data or simply touch data), and temporarily storing the time-series data in the touch data storage unit 31.

The work segmentation detection processing unit 13 reads the touch time-series data from the touch data storage unit 31 and detects a segmentation of each work section from the read touch time-series data. Then, processing for storing the touch time-series data of each of the segmented work sections as the work-by-work touch time-series data in the work-by-work touch data storage unit 32 is performed. An example of a method of detecting the segmentation of the work section will be described in an operation example.

The touch time histogram generation processing unit 14 reads the touch time-series data for each work from the work-by-work touch data storage unit 32 for each work, and generates a touch time histogram based on the read touch time-series data for each work. The touch time histogram indicates a frequency distribution of the touch time, that is, a distribution of the number of touches per touch time.

The work type estimation processing unit 15 estimates a type of work from a pattern of a touch time frequency distribution indicated by the touch time histogram for each work. For each work, processing is performed to generate work history information including the estimated type of work and to store the generated work history information in the work history information storage unit 34.

When the work history information is generated, the work type estimation processing unit 15 acquires a worker ID and a work target ID included in the inter-human body communication signal, and a start time and an end time of each work obtained from a clock (not illustrated) during detection of the work segmentation, and includes the worker ID, the work target ID, the start time, and the end time in the work history information. An example of a method of estimating a type of work will be described in the operation example.

The work history information output processing unit 16 performs processing for reading corresponding work history information from the work history information storage unit 34 in accordance with output instruction content of the work history information input from the input device 51, editing or not editing the work history information, displaying the edited or unedited work history information on the display device 52, and transmitting the work history information to the server device SV.

### (Operation Example)

Next, an operation example of the work information processing device WT that has the foregoing configuration will be described.

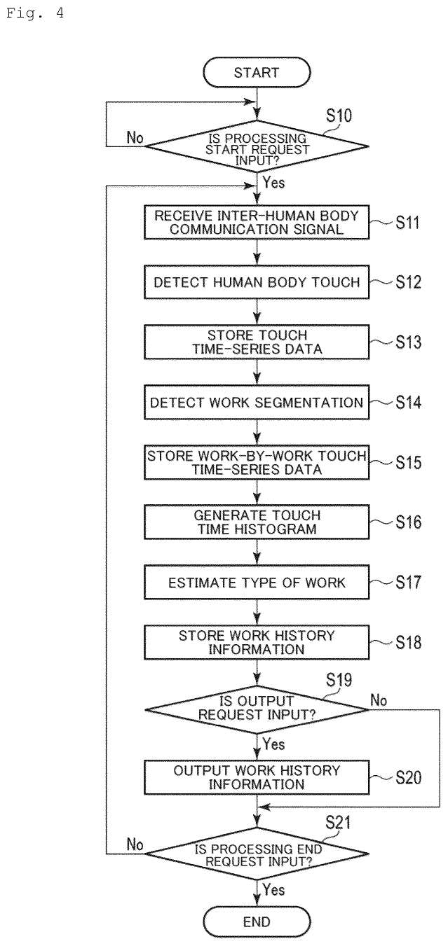

FIG. 4 is a flowchart illustrating an example of a processing order and processing content of a series of processes by the work information processing device WT.

### (1) Generating Touch Time-Series Data

The control unit 1 of the work information processing device WT monitors an input of an operation start request in step S10. In this state, for example, when the worker NU inputs the operation start request using the input device 51, the control unit 1 subsequently starts generation processing for the work history information.

That is, under the control of the inter-human body communication signal acquisition processing unit 11, the control unit 1 of the work information processing device WT receives the inter-human body communication signal transmitted from the communication terminals T_{NU} and T_{PU} of the inter-human body communication system HC via the communication I/F unit 4 and converts the received inter-human body communication signal into digital data in step S11. Then, the digital data of the inter-human body communication signals are delivered to the touch detection processing unit 12.

The control unit 1 of the work information processing device WT subsequently detects a period in which a human body is touched from the inter-human body communication signals under the control of the touch detection processing unit 12 in step S12. The detection of the touch period is performed, for example, by detecting a period in which an amplitude of the inter-human body communication signal is equal to or higher than a given level. The touch detection processing unit 12 stores touch time-series data indicating the detected touch period in the touch data storage unit 31 in step 313.

### (2) Detecting Work Section

The control unit 1 of the work information processing device WT subsequently detects a segmentation of each work section from the touch time-series data under the control of the work segmentation detection processing unit 13 in step S14. The detection of the segmentation of the work section is performed by detecting, for example, a non-work period equal to or longer than a fixed length.

That is, in general, when a nursing or care operation is performed, a plurality of operations are not continuously performed, and an interval period is often included between types of work. When there is a period in which a touch does not continue for a fixed time or longer, the work segmentation detection processing unit 13 detects this period as the interval period and sets this period as a segmentation of the work.

The detection of the work segmentation may be performed by using data learned in advance by, for example, recurrent neural network (RNN) machine learning. This method is, for example, a method of learning a time-series distribution pattern of the touch for all assumed operations and using this section as a segmentation of the work when a pattern other than the distribution pattern is detected.

When the segmentation of the work section is detected, the work segmentation detection processing unit 13 stores touch time-series data for each partitioned work section as work-by-work touch time-series data in the work-by-work touch data storage unit 32 in step S15.

### (3) Generating Touch Time Histogram

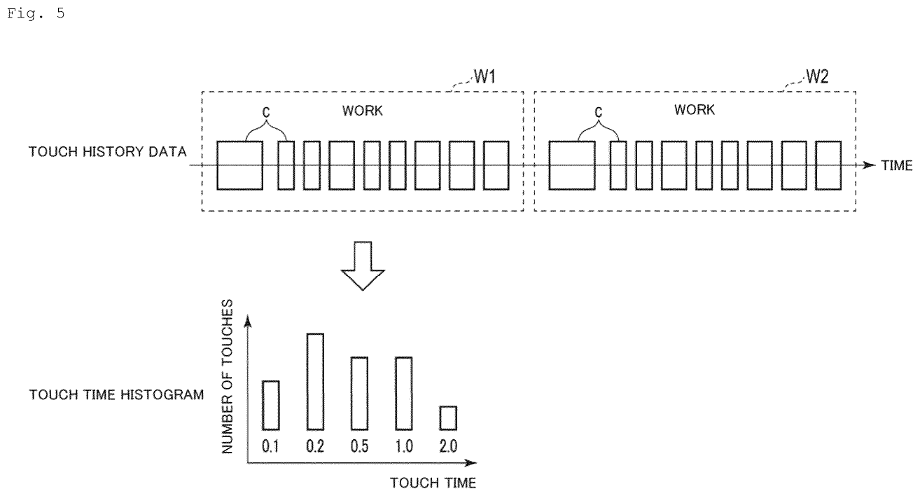

The control unit 1 of the work information processing device WT subsequently reads the work-by-work touch time-series data from the work-by-work touch data storage unit 32 under the control of the touch time histogram generation processing unit 14 in step S16. Then, a touch time histogram indicating a distribution of the number of touches for each touch time is generated for each work by statistically processing the read work-by-work touch time-series data. FIG. 5 illustrates an example of the operation, and illustrates a case where the distribution of the number of touches for each time of each touch included in the work section is obtained for work W1 between the works W1 and W2 partitioned through the work section detection processing. A touch time histogram is similarly generated for work W2.

### (4) Estimating Type of Work

In step S17, the control unit 1 of the work information processing device WT subsequently estimates a type of work from the pattern of the touch time frequency distribution indicated by the touch time histogram for each work under control of the work type estimation processing unit 15.

In this estimation processing, for example, learned data of the work model generated in advance by convolutional neural network (CNN) machine learning is stored in the work model storage unit 33. A method of estimating a type of work is used based on the learned data of the work model stored in the work model storage unit 33. As the method of estimating the type of work, for example, a method of comparing the pattern of the touch time distribution of the touch time histogram with a reference pattern of a histogram of each work acquired and stored in advance and selecting a type of work in which the difference is minimum can also be applied.

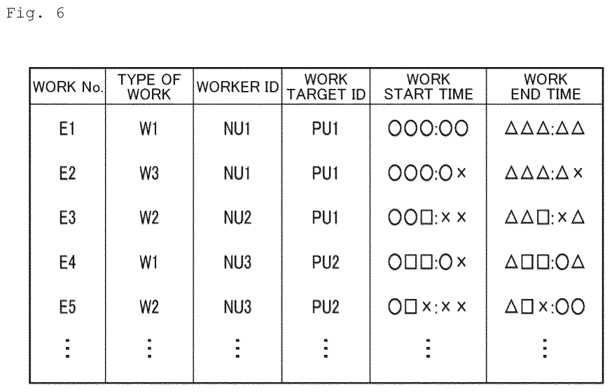

In step S18, the work type estimation processing unit 15 subsequently generates work history information for each work and stores the generated work history information in the work history information storage unit 34. FIG. 6 is a diagram illustrating an example of a configuration of the work history information. In this example, the work history information includes a number for each work, the estimated work type, a worker ID and a work target ID extracted from the inter-human body communication signal, the start time and the end time of each work obtained from a clock (not shown) when detecting the work segmentation are included.

### (5) Outputting Work History Information

In step S19, the control unit 1 of the work information processing device WT monitors an input of an output request. In this state, when the worker NU inputs, for example, a display request of work history information in an input device 51, the control unit 1 of the work information processing device WT reads corresponding work history information from the work history information storage unit 34, outputs the display data of the read work history information from the input/output I/F unit 5 to the display device 52, and displays the display data on the display device 52 under the control of the work history information output processing unit 16 in step S20.

At this time, the work history information output processing unit 16 may display the work history information as it is, but may extract and display only data associated with the same worker ID or extract and display only the data associated with the same work target ID, for example, in response to an operation of the worker NU in a display state. In addition, it is also possible to extract and display only data of which the types of work are the same or to specify, for example, a date or a time period and extract and display only data within the range.

The work history information output processing unit 16 can transmit the work history information from the communication I/F unit 4 to the server device SV in response to an operation of the worker NU or can transmit the work history information from the communication I/F unit 4 to the terminal of the work target PU or her or his family after appropriately editing the work history information.

### (Operations and Effects)

As described above, the work information processing device WT according to the embodiment receives the inter-human body communication signal output from the inter-human body communication system HC in accordance with a nursing or care operation. Then, the touch time-series data is extracted for each work by first generating the time-series data of the touch during the work from the received inter-human body communication signal and detecting the segmentation of the work from the touch time-series data. Subsequently, a touch time histogram indicating a feature of a touch pattern in a work section is generated from the touch time-series data, a type of work is estimated based on the touch time histogram, and work history information including information indicating the estimated type of work is generated.

Accordingly, not only presence or absence of a touch accompanying the work of the worker but also the type of work is estimated and recorded in the work history information based on the inter-human body communication signal. Accordingly, the operation of recording the type of work by the worker is not required, and a work load of the worker can be reduced.

For each work, the worker ID and the work target ID are extracted from the inter-human body communication signal at that time, and a start time and an end time of the work are generated and included in the work history information. Therefore, in the work history information, the worker ID, the work target ID, and the start time and the end time of the work are automatically recorded in addition to the type of work. Accordingly, the load of the operator is further reduced.

Further, the touch time histogram is used when the feature amount of the touch pattern is detected for each work. Therefore, the feature amount of the touch pattern can be detected through relatively simple processing. Accordingly, it is possible to reduce a processing load of a control unit required for estimation processing of the type of work and shorten a processing time.

### [Other Embodiments]

In the foregoing embodiment, the case where a personal computer or the like provided at a work site is used as the work information processing device WT has been described as an example. However, the present invention is not limited thereto. For example, all or some of the functions of the work information processing device WT according to the present invention may be provided in the server device SV on a cloud or a web. In this case, the personal computer at the site operates as a relay terminal transferring the inter-human body communication signal to the server device.

In the foregoing embodiment, the case where the type of work is estimated from the inter-human body communication signal and recorded in the work history information has been described as an example. However, the present invention is not limited thereto. For example, a work time length for each type of work is compared with a standard work time length generated by pre-learning or the like, the quality of work, for example, the degree of accuracy, is estimated from a time difference, and the result may be recorded in the work history information. Work efficiency may be determined based on the time difference of the work time length, and the result may be recorded in the work history information.

In the foregoing embodiment, the case in which the type of work at the nursing or nursing site is estimated has been described as an example. However, the present invention can also be applied to an instruction site such as chiropractic, acupuncture and moxibustion, massage, hairdressing and beauty, dancing and gymnastics. In addition, for a type or configuration of the work information processing device, content or the like of work segmentation detection processing, extraction processing of a touch feature amount, and estimation processing of a type of work, various modifications can be made without departing from the gist of the present invention.

While the embodiments of the present invention have been described in detail, the above-described description is merely an example of the present invention in all respects. It is needless to say that various improvements and modifications can be made without departing from the scope of the present invention. That is, when the present invention is embodied, a specific configuration corresponding to an embodiment may be adopted appropriately.

In other words, this invention is not limited to the embodiment as it is but can be embodied by modifying components in the practical phase without departing from the gist thereof. Also, various inventions can be embodied by appropriately combining a plurality of constituents disclosed in the foregoing embodiment. For example, some of all the constituents described in the embodiment may be omitted. Furthermore, components of different embodiments may be combined as appropriate.

### REFERENCE SIGNS LIST

WT Work information processing device
HC Inter-human body communication system
T_{NU}, T_{PU} Communication terminal
NW Network
SV Server device
1 Control unit
2 Program storage unit
3 Data storage unit
4 Communication i/f unit
5 Input/output i/f
6 Bus
11 Inter-human body communication signal acquisition unit
12 Touch detection processing unit
13 Work segmentation detection processing unit
14 Touch time histogram generation unit
15 Work type estimation processing unit
16 Work history information output processing unit
31 Touch data storage unit
32 Work-by-work touch data storage unit
33 Work model storage unit
34 Work history information storage unit

1. A work information processing device for connection to an inter-human body communication system outputting an inter-human body communication signal transmitted between a first person and a second person when the first person and the second person touch each other in work, the work information processing device comprising:
touch detection processing circuit configured to receive the inter-human body communication signal output from the inter-human body communication system, to detect a time-series position of the touch based on the received inter-human body communication signal, and to generate touch time-series data indicating a detection result;
work section detection processing circuitry configured to detect a work section in which the work is performed from the touch time-series data and to extract the touch time-series data included in the detected work section;
touch feature amount detection processing circuity configured to detect a feature amount of the touch in the work section based on the touch time-series data included in the work section;
estimation processing circuitry configured to estimate a type of work based on the detected feature amount of the touch, and
work history information generation processing circuitry configured to generate work history information including information indicating the estimated type of work for each work.

2. The work information processing device according to claim 1, wherein:
the work history information generation processing circuitry extracts identification information of the first person and the second person from the inter-human body communication signal and generates the work history information further including the extracted identification information of the first person and the second person for each of the work

3. The work information processing device according to claim 1, wherein;
the work history information generation processing circuitry generates information indicating a start time and an end time of the work section and generates the work history information further including generated information indicating the start time and the end time of the work section.

4. The work information processing device according to claim 1, wherein:
the touch feature amount detection processing circuitry detects a touch time frequency distribution indicating the number of touches for each touch time in the work section based on the touch time-series data included in the work section.

5. A work information processing method, comprising:
receiving a inter-human body communication signal output from a inter-human body communication system, detecting a time-series position of the touch based on the received inter-human body communication signal, and generating touch time-series data indicating a detection result;
detecting a work section in which work is performed from the touch time-series data and extracting the touch time-series data included in the detected work section;
detecting a feature amount of the touch in the work section based on the touch time-series data included in the work section;
estimating a type of work based on the detected feature amount of the touch, and
generating work history information including information indicating the estimated type of work for each work.

6. A non-transitory computer readable medium storing a program for causing a processor included in the work information processing device according to claim 1 to execute processing of the circuitries included in the work information processing device.

7. A non-transitory computer readable medium storing a program for causing a processor to perform the method of claim 5.
